(19) United States
(12) Patent Application Publication
(10) Pub. No.: US 2023/0081566 A1
(43) Pub. Date: Mar. 16, 2023

(54) SYSTEMS AND METHODS FOR PREDICTING MYOPIA RISK

(71) Applicant: Johnson & Johnson Vision Care, Inc., Jacksonville, FL (US)

(72) Inventors: Noel Brennan, Jacksonville, FL (US); Mark Bullimore, Boulder, CO (US)

(21) Appl. No.: 17/466,132

(22) Filed: Sep. 3, 2021

(51) Int. Cl. G16H 50/30; G16H 10/60; G16H 50/70; G16H 50/50; G16H 50/20

(52) U.S. Cl. CPC G16H 50/30; G16H 10/60; G16H 50/70; G16H 50/50; G16H 50/20; A61B 5/7275

(57) ABSTRACT

A system and method including receiving, via an interface, demographic information and behavioral information associated with a subject; determining an incidence factor for the subject by weighting, according to a predetermined incidence formula, the demographic information and the behavioral information, wherein the predetermined incidence formula and weighting is derived from incidence data associated with a population; determining a progression factor for the subject by weighting, according to a predetermined progression formula, the demographic information and the behavioral information, wherein the predetermined progression formula and weighting is derived from progression data associated with a population, and wherein the predetermined progression formula is a function of the incidence factor; predicting and calculating based on the incidence factor and the progression factor, a myopia risk metric indicative of risk of the subject exhibiting myopia; and outputting the myopia risk metric as a numerical component.

### BACKGROUND

Common conditions which lead to reduced visual acuity include myopia and hyperopia, for which corrective lenses in the form of spectacles, or rigid or soft contact lenses, are prescribed. The conditions are generally described as the imbalance between the length of the eye and the focus of the optical elements of the eye. Myopic eyes focus light in front of the retinal plane and hyperopic eyes focus light behind the retinal plane. Myopia typically develops because the axial length of the eye grows to be longer than the focal length of the optical components of the eye, that is, the eye grows too long. Hyperopia typically develops because the axial length of the eye is too short compared with the focal length of the optical components of the eye, that is, the eye does not grow long enough.

Myopia has a high prevalence rate in many regions of the world. Of greatest concern with this condition is its possible progression to high myopia, for example, greater than five (5) or six (6) diopters, which dramatically affects one's ability to function without optical aids. High myopia is also associated with an increased risk of retinal disease, cataract, glaucoma, and myopic macular degeneration (MMD; also known as myopic retinopathy), and may become a leading cause of permanent blindness worldwide. For example, MMD has been related to refractive error (RE) to a degree rendering no clear distinction between pathological and physiological myopia and such that there is no “safe” level of myopia.

Corrective lenses are used to alter the gross focus of the eye to render a clearer image at the retinal plane, by shifting the focus from in front of the plane to correct myopia, or from behind the plane to correct hyperopia, respectively. However, the corrective approach to the conditions does not address the cause of the condition, but is merely prosthetic or intended to address symptoms.

Some applications and websites have been developed to provide general guidance relating to myopia risk. For example, https://www.mykidsvision.orglen-US provides a questionnaire and generic categorical feedback (e.g., low risk, medium risk, high risk). As another example, https://coopervision.com/eye-health-and-vision/childhood-short-sightedness/assessment-tool also provides a questionnaire and generic categorical feedback (e.g., low risk, medium risk, high risk). As a further example, Myappia (https://play.google.com/store/appsidetails?id=:com myappia.myappia&hl=°°es EC) is a software tool for visualizing the expected progression of myopia or nearsightedness over time. Myappia allows for the entry of the patient's age and initial prescription and then calculates based on several longitudinal studies, the likely progression of myopia over the following 10 years based on comparative treatment curves such as standard glasses and contact lenses as well as with your choice of bifocal spectacles, progressive addition multifocals, “flat optical profile” contact lenses, low dose atropine, bifocal contact lenses, orthokeratology and custom myopia control contact lenses. There are certain assumptions associated with each treatment choice from an average of the studies available and these percentage reductions are used to modify the predicted progression curves.

Improvements over prior art tools are needed, particularly in terms of more precise, quantitative indicators of future myopia risk, such as for example a percentage, as opposed to the more general, qualitative prediction outputs currently provided.

### SUMMARY

A system and computer-implemented system is provided including the steps of receiving, via an interface, demographic information indicative of an age of a subject, a gender of the subject, an ethnicity of the subject, and a number of myopic parents of the subject receiving, via the interface, behavioral information indicative of a time that the subject spends outside each day and a time that the subject spends on nearwork each day; determining an incidence factor for the subject by weighting, according to a predetermined incidence formula, the demographic information and the behavioral information, wherein the predetermined incidence formula and weighting is derived from incidence data associated with a population; determining a progression factor for the subject by weighting, according to a predetermined progression formula, the demographic information and the behavioral information, wherein the predetermined progression formula and weighting is derived from progression data associated with a population, and wherein the predetermined progression formula is a function of the incidence factor; predicting and calculating, by a processor and based on the incidence factor and the progression factor, a myopia risk metric indicative of risk of the subject exhibiting myopia; and causing output of the myopia risk metric comprising a quantitative numerical component, which may be a percentage.

According to one embodiment, the incidence factor is based at least on the following formulaic relationship: incidence factor=BI x G x a x E x β^{l1}′, where BI is a baseline incidence factor, G is a gender weighting factor, E is an ethnicity weighting factor, MP is the number of myopic parents of the subject and a and β are evidence-based weighting factors.

According to another embodiment, the incidence factor is based at least on the following formulaic relationship: incidence factor=BI x (1+G×0.15)×E×1.6^{MP-1}where BI is a baseline incidence factor, G is a gender weighting factor, E is an ethnicity weighting factor, MP is the number of myopic parents of the subject.

According to various embodiment, BI may be 0.04, G may be 1 for female and 0 for male, and/or E may be 2.5 for Asians, 2 for Hispanics, and 1 for others.

In yet another embodiment, the incidence factor is based at least on the following formula: incidence factor=BI x (1+G×0.15)×E×1.6^{MP-1} ×0.5^{OT-1} ×1.1 ^{NT-1}, where BI is a baseline incidence factor, G is a gender weighting factor, E is an ethnicity weighting factor, MP is the number of myopic parents of the subject, OT is the time (hours) the subject spends outdoors each day, and NT is the time (hours) the subject spends on nearwork each day.

BI may be 0.04, G may be 1 for female and 0 for male, and/or E may be 2.5 for Asian, 2 for Hispanic, and 1 for others.

In one embodiment, the progression factor indicates a probability of the subject exhibiting high myopia, wherein high myopia is at least −4D.

In another embodiment, the progression factor for a subject having ethnicity of Asian is based on at least the following formula: progression factor=incidence factor x 10^{2.1-0.293 xA} ×(0.9+0.1 ×MP^{1.5})×(0.98+0.02 ×G), where A is age in years of the subject, G is a gender weighting factor, and MP is the number of myopic parents of the subject. G may be 1 for female and 0 for male.

In yet another embodiment, the progression factor for a subject having ethnicity of non-Asian is based on at least the following formula: progression factor=incidence factor x 10^{1.37-0.293 ×A} ×(0.9+0.1 ×MP^{1.5})×(0.98+0.02 ×G), where A is age in years of the subject, G is a gender weighting factor, and MP is the number of myopic parents of the subject. G may be 1 for female and 0 for male.

According to another embodiment, the method further includes receiving diagnostic information indicative of one or more of a refractive error associated with the subject or axial length of an eye of the subject indicating that the subject is non-myopic, wherein one or more of the incidence factor or the progression factor is determined based on at least the diagnostic information.

According to various embodiments, the risk metric may be a severity metric, and the severity metric may be a projected level of myopia.

A device and system may be configured to implement the methods.

Also provided is a computer-implemented method including the steps of receiving, via an interface, demographic and behavioral information of a subject; determining based on this information an incidence factor for the subject by weighting, according to a predetermined incidence formula, the demographic information, and behavioral information, wherein the predetermined incidence formula and weighting is derived from incidence data associated with a population; determining based on one or more of the demographic or behavioral information, a progression factor for the subject by weighting, according to a predetermined progression formula, the demographic and behavioral information, wherein the predetermined progression formula and weighting is derived from progression data associateds with a population, and wherein the predetermined progression formula is a function of the incidence factor; predicting and calculating, by a processor and based on the incidence factor and the progression factor, one or more of a myopia risk metric indicative of risk of the subject exhibiting myopia or a severity metric indicative of a level of myopia; and causing output of the one or more of the myopia risk metric or the severity metric, wherein each of the myopia risk metric and the severity metric comprises a quantitative numerical component, which may be a percentage.

The demographic information may be age of the subject, gender of the subject, ethnicity of the subject and/or the number of myopic parents of the subject.

The behavioral information may be the time the subject spends outside each day and/or the time the subject spends on nearwork each day.

According to another embodiment, the method further includes the step of receiving, via the interface, measurable diagnostic information indicative of one or more of refractive error associated with the subject or axial length of an eye of the subject, wherein the predicting and calculating step is based at least in part on the measurable diagnostic information.

The incidence factor may be based on at least the following formulaic relationship of incidence factor=BI ×G×α×E×β^{MP}, where BI is a baseline incidence factor, G is a gender weighting factor, E is an ethnicity weighting factor, MP is the number of myopic parents of the subject and a and ii are evidence-based weighting factors.

Alternatively, the incidence factor may be based at least on the following formula: incidence factor=BI ×(1+G×0.15)×E×1.6^{MP-1} where BI is a baseline incidence factor, G is a gender weighting factor, E is an ethnicity weighting factor, MP is the number of myopic parents of the subject.

BI may be 0.04, G may be 1 for female and 0 for male and/or E may be 2.5 for Asian, 2 for Hispanic and 1 for others.

In yet another alternative embodiment, the incidence factor may be based at least on the following formula: incidence factor=BI ×(1+G×0.15)×E×1.6^{MP-1} ×0.5^{OT-1} ×1.1^{NT-1}, where BI is a baseline incidence factor, G is a gender weighting factor, E is an ethnicity weighting factor, MP is the number of myopic parents of the subject, OT is the time (hours) the subject spends outdoors each day, and NT is the time (hours) the subject spends on nearwork each day.

BI may be 0.04, G may be 1 for female and 0 for male, and/or E may be 2.5 for Asian, 2 for Hispanic and 0 for others.

The progression factor may indicate a probability of the subject exhibiting high myopia (at least -5D) through 18 years of age of the subject.

In one embodiment, the progression factor for a subject having an ethnicity of Asian is based at least on the following formula: progression factor=incidence factor x 10².14293 ^{xA}×(0.9+0.1 ×MP^{1.5})×(0.98+0.02 ×G), where A is age in years of the subject, G is a gender weighting factor, and MP is the number of myopic parents of the subject. G may be 1 for female and 0 for male.

In another embodiment, the progression factor for a subject having ethnicity of non-Asian is based on at least the following formula: progression factor=incidence factor x 10^{1.37-0.293} ×A ×(0.9+0.1 ×MP^{1.5})×(0.98+0.02 ×G), where A is age in years of the subject, G is a gender weighting factor, and MP is the number of myopic parents of the subject. G may be 1 for female and 0 for male.

The myopia severity metric may be a projected level of myopia.

The risk metric may be a severity metric, and the myopia severity metric may be a projected level of myopia.

Also provided herein is a device and/or system configured to implement the methods described herein.

### BRIEF DESCRIPTION OF THE DRAWINGS

The following drawings show generally, by way of example, but not by way of limitation, various examples discussed in the present disclosure. In the drawings:

FIG. 1 depicts a computer-implemented system for assessing a myopia risk of an individual.

FIG. 2 depicts a method for assessing myopia risk according to one embodiment.

FIG. 3 shows a representative hardware environment for practicing at least one embodiment of the present invention.

FIG. 4 shows prior art example data on the youngest age at which myopia was observed.

FIG. 5 shows prior art example data of annual incidence of myopia and high myopia in primary and junior high school cohorts based on refraction without cycloplegia.

FIG. 6 shows prior art example plot of age specific incidence of myopia indicating that incidence is relatively constant with age.

FIG. 7 shows a prior art logistic model showing results of inclusion of all significant variables from the AUC models adjusted for other variables.

FIG. 8 shows prior art plot of parental history data.

FIG. 9 shows prior art data relating to modeled hazard ratios for the development of myopia.

FIG. 10 shows a prior art data plot relating to outdoor activity.

FIG. 11 shows a prior art data plot relating to outdoor activity and nearwork.

FIG. 12 shows survival probability curves for no, one, and two myopic parents as a function of risk group.

FIG. 13 shows hazard ratio (HR) for myopia incidence.

FIG. 14 shows an extract of the data presented in a prior art study.

FIG. 15 shows data illustrating the relation between baseline refractive error and the incidence of myopia.

FIG. 16 shows prior art data relating to the risk of high myopia in adulthood, stratified by sex and age at myopia onset.

FIGS. 17A, 17B, and 17C show prior art data illustrating the risk of developing high myopia as a function of age.

FIG. 18 shows a comparison of the risk of high myopia as a function of age of onset in Asian and European myopes.

FIG. 19 shows a comparison of the risk of high myopia at 25 years and 18 years as a function of age of onset in European myopes.

### DETAILED DESCRIPTION

Disclosed herein are systems and methods, such as computer-implemented methods, for assessing myopia risk. The systems and methods of the present disclosure provide a quantitative myopia risk metric as an output that is a numerical value as opposed to a few large general categories of risk such as “low”, “medium” and “high.” The myopia risk metric is based on a subject's fixed factors, behavioral factors, and optionally measurable diagnostic factors which are assessed in a unique manner leveraging population data as will be described in further detail below. Measurable diagnostic factors are considered optional as the present system and method has applicability to, and is useful in different settings and with different target users. A first setting may be a home setting in which a parent is interested in acquiring a myopia risk assessment for a child when diagnostic measurements are not available and/or have not been previously obtained. A second setting may be an eye practitioners office or the like, where diagnostic measurements can be obtained or past measurements stored or kept for ready access.

The system and method of the present invention will be described herein, first generally with regard to a computing system on which it can be implemented along with the various subject specific information that can be provided as input data, and subsequently the population data that is leveraged and the formula(s) and weighting applied to that population data to produce a quantitative numerical value indicative of myopia risk for that subject.

As noted, the system and method described herein can be implemented on a computer system where information specific to a subject can be provided as input. Subject provided input will include demographic or fixed variable input information, behavioral input information and optionally measurable diagnostic input information. Demographic or fixed variable information refers to factors that may vary across a population, but are fixed relative to a given individual, such as age, a gender, ethnicity, and the number of myopic parents of the subject. Behavioral variable information is not fixed relative to a given subject, but rather is subject to modification if desired. Such behavioral information may include the time that the subject spends outside each day and the time that the subject spends on nearwork each day. Measurable diagnostic information are measurable characteristics of the particular subject, and may include refractive error associated with the subject or an axial length of an eye of the subject.

The demographic, behavioral and optionally measurable diagnostic information can be used in conjunction with population data to calculate a more precise myopia risk metric. As will be described further below, an incidence factor is determined for the subject by weighting according to a predetermined incidence formula, the demographic, behavioral and optional measurable diagnostic information input by the subject, where the weighting and predetermined incidence formula are derived from incidence data associated with a population. A progression factor for the subject is also determined from the demographic, behavioral and optional measurable diagnostic information, where the weighting and predetermined progression formula is derived from progression data associated with a population and where the progression formula is a function of the incidence factor. The incidence factor and progression factor are then used to generate a quantitative myopia risk metric, such as a numerical value. The myopia risk metric may include a severity metric, which may indicate a level of projected myopia severity (e.g., −2D, −4D, −6D, −7D, −8D, etc.).

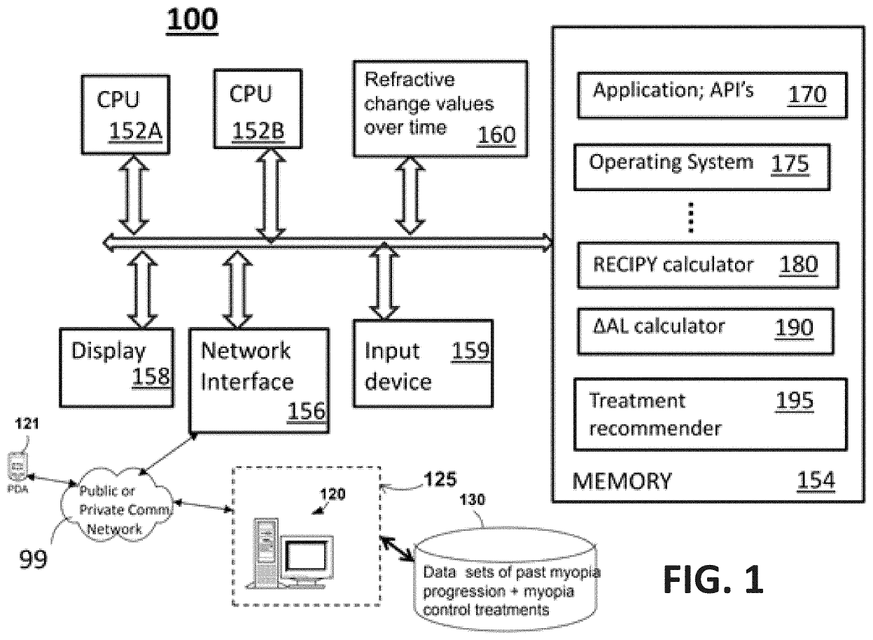

FIG. 1 depicts an exemplary computer-implemented system 100 for predicting myopia risk (e.g., myopia incidence, myopia progression, etc.), which may include any well-known type of computing device, such as personal computers, laptops, tablets, smart devices, smart phones, servers or any other similar computing device (or combination thereof) for receiving input data; for performing data analysis such as one or more of the method steps discussed herein, and for outputting data. The input data and output data may be stored or saved in at least one database 130. The input and/or output data may be accessed by a software application 170 installed on the computer system 100 (for example a computer in the office of an Eye Care Practitioner (ECP) or in the home of an individual or subject); by a downloadable software application (app) on a smart device 121; or by a secure website 125 or web link accessible by a computer via network 99. The input and/or output data may be displayed on a graphical user interface of a computer or smart device.

In particular, computing system 100 includes one or more hardware processors 152A, 152B, a memory 154, e.g., for storing an operating system and application program instructions, a network interface 156, a display device 158, an input device 159, and any other features common to a computing device. The computing system 100 may be configured to communicate with a web-site 125 or web- or cloud-based server 120 over a public or private communications network 99. Further, as shown as part of system 100, historical data pertaining to individuals' refractive changes captured from clinicians' measurements and including associated myopia control treatments, are obtained and stored in an attached, or a remote memory storage device, e.g., a database 130.

In the embodiment depicted in FIG. 1, processors 152A, 152B may include, for example, a microcontroller, Field Programmable Gate Array (FPGA), or any other processor that is configured to perform various operations, and may be configured to execute instructions as described below. These instructions may be stored, for example, as programmed modules in memory storage device 154.

Memory 154 may include, for example, non-transitory computer readable media in the form of volatile memory, such as random access memory (RAM) and/or cache memory or others, or other removable/non-removable, volatile/non-volatile storage media. By way of non-limiting examples only, memory 154 may include a portable computer diskette, a hard disk, a random access memory (RAM), a read-only memory (ROM), an erasable programmable read-only memory (EPROM or Flash memory), a portable compact disc read-only memory (CD-ROM), an optical storage device, a magnetic storage device, or any suitable combination of the foregoing.

Network interface 156 is configured to transmit and receive data or information to and from a web-site server 120, e.g., via wired or wireless connections. For example, network interface 156 may utilize wireless technologies and communication protocols such as Bluetooth®, WIFI (e.g., 802.11a/b/g/n), cellular networks (e.g., CDMA, GSM, M2M, and 3G/4G/4G LTE), near-field communications systems, satellite communications, via a local area network (LAN), via a wide area network (WAN), or any other form of communication that allows computing device 100 to transmit information to or receive information from the server 120.

Display 158 may include, for example, a computer monitor, television, smart television, a display screen integrated into a personal computing device such as, for example, laptops, smart phones, smart watches, virtual reality headsets, smart wearable devices, or any other mechanism for displaying information to a user. In some aspects, display 158 may include a liquid crystal display (LCD), an e-paper/e-ink display, an organic LED (OLED) display, or other similar display technologies, and may be touch-sensitive and may also function as an input device.

Input device 159 may include, for example, a keyboard, a mouse, a touch-sensitive display, a keypad, a microphone, or other similar input devices or any other input devices that may be used alone or together to provide a user with the capability to interact with the computer system 100.

With respect to the ability of computer system 100 for computing a myopia risk metric, the system 100 includes: a memory 160 configured to store data which could optionally include data relating to a current individual's past refractive changes/errors, e.g., data received from a clinician over a defined period of time, e.g., a past year. In one embodiment, this data may be stored in a local memory 160, i.e., local to the computer or mobile device system 100, or otherwise, may be retrieved from a remote server 120, over a network. The data relating to a current individual's past refractive changes may be accessed via a remote network connection for input to a local attached memory storage device 160 of system 100.

In one embodiment, the computing system 100 provides a technology platform employing programmed processing modules stored in a device memory 154 that may be run via the processor(s) 152A, 152B to provide the system with abilities for predicting myopia risk (e.g., calculating a myopia risk metric such as a myopia risk metric comprising a numerical component.

In one embodiment, program modules stored in memory 154 may include operating system software 170 and a software applications module 175 for running the methods herein that may include associated mechanisms such as APIs (application programming interfaces) for specifying how the various software modules interact, web-services, etc. that are employed to control operations used to carry out predicting myopia risk. One program module 180 stored in device memory 154 may include a “RECIPY” calculator 190 for determining a value (“RECIPY”) representative of a current individual's refractive change in a past time period, e.g., one year. From this RECIPY refractive rate of change value of the individual, a further program module 190 stored in device memory 154 may include program code providing the various data and processing instructions of an algorithm that is run by the processors to predict a change in axial length (“ΔAL”) value for that individual. Based on the predicted change in axial length (“ΔAL”) value for that individual, a further module 195 may be invoked to output to a clinician, the individual, or any user, a myopia risk metric such as a myopia risk metric comprising a numerical component.

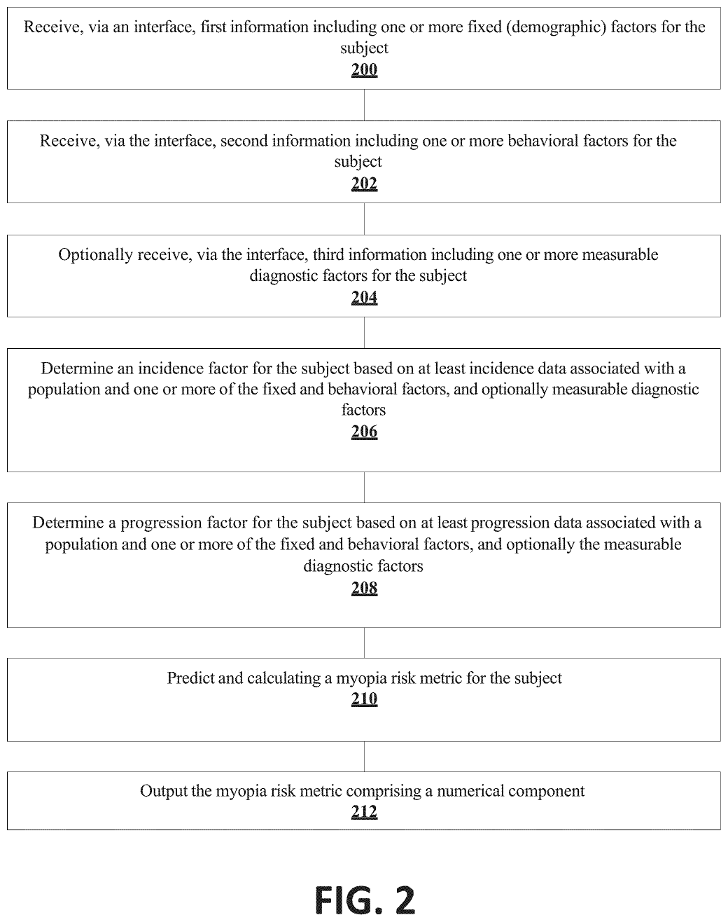

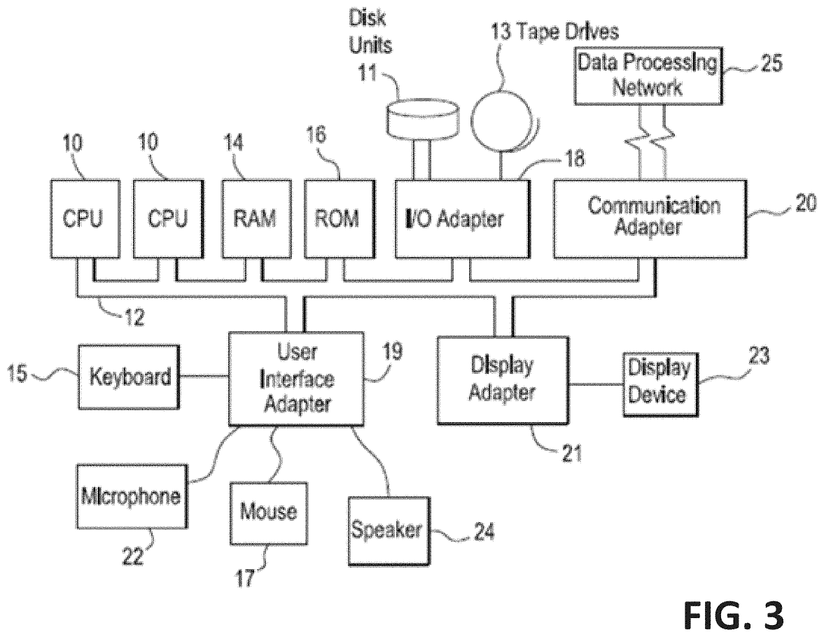

FIG. 2 depicts a method employed for assessing myopia risk according to one embodiment that may be implemented via the system 100 of FIG. 1. At 200, first information may be received via an interface (i.e., 22, 17 or 24 of FIG. 3). The first information may be fixed or demographic information (used interchangeably herein) indicative of, for example, an age of a subject, a gender of the subject, an ethnicity of the subject, and a number of myopic parents of the subject.

At 202, second information may be received via an interface. The second information may include behavioral information such as the time that the subject spends outside each day and the time that the subject spends on nearwork each day.

At 204, third information may optionally be received via the interface. The third information may comprise measurable diagnostic information such as refractive error associated with the subject or axial length of an eye of the subject.

At 206, an incidence factor for the subject is determined by weighting, according to a predetermined incidence formula, the information received in steps 200, 202 and optionally 204. The predetermined incidence formula and weighting is derived from data associated with a population.

In one example, the incidence factor is based at least on the following incidence formula: incidence factor=BI ×(1+G×0.15)×E×1.6^{MP-1}, where BI is a baseline incidence factor, G is a gender weighting factor, E is an ethnicity weighting factor, and MP is the number of myopic parents of the subject. BI may be 0.04. G may be 1 for female and 0 for male. E may be 2.5 for Asian, 2 for Hispanic, 1 for others. Other weights (values) may be derived and used.

In another example, the incidence factor is based at least on the following incidence formula: incidence factor=BI ×(1+G×0.15)×E×1.6^{MP-1}×0.5^{OT-1} ×1.1^{NT-1}, where BI is a baseline incidence factor, G is a gender weighting factor, E is an ethnicity weighting factor, MP is the number of myopic parents of the subject, OT is the time (hours) the subject spends outside each day, and NT is the time (hours) the subject spends on nearwork each day. BI may be 0.04. G may be 1 for female and 0 for male. E may be 2.5 for Asian, 2 for Hispanic, 1 for others. Other weights (values) may be derived and used.

At 208, a progression factor is determined for the subject by weighting, according to a predetermined progression formula and the information received in steps 200, 202 and optionally 204. The progression factor may indicate a probability of the subject exhibiting high myopia (at least −5D) through 18 years of age of the subject.

The progression factor for a subject having ethnicity of Asian may be based on at least the following formula: progression factor=incidence factor x₁₀2.1-0.293. A ×(0.9+0.1 ×MP^{1.5})×(0.98+0.02 ×G), where A is age in years of the subject, G is a gender weighting factor, and MP is the number of myopic parents of the subject. G may be 1 for female and 0 for male. Other weights (values) may be derived and used.

The progression factor for a subject having ethnicity of non-Asian may be based on at least the following formula: progression factor=incidence factor x₁₀1.37-0.293. A ×(0.9+0.1 ×MP^{1.5})×(0.98+0.02 ×G), where A is age in years of the subject, G is a gender weighting factor, and MP is the number of myopic parents of the subject. G may be 1 for female and 0 for male. Other weights (values) may be derived and used.

At 210, a myopia risk metric indicative of risk of the subject exhibiting myopia is predicted and calculated by a processor and based on the incidence factor and the progression factor.

At 212, the myopia risk metric as a numerical component is provided as an output.

A device and/or system may be configured to implement the method depicted in FIG. 2.

Referring now to FIG. 3, a representative hardware environment for practicing at least one embodiment of the invention is depicted. This schematic drawing illustrates a hardware configuration of an information handling/computer system in accordance with at least one embodiment of the invention. The system comprises at least one processor or central processing unit (CPU) 10. The CPUs 10 are interconnected with system bus 12 to various devices such as a random access memory (RAM) 14, read-only memory (ROM) 16, and an input/output (I/O) adapter 18. The I/O adapter 18 can connect to peripheral devices, such as disk units 11 and tape drives 13, or other program storage devices that are readable by the system. The system can read the inventive instructions on the program storage devices and follow these instructions to execute the methodology of at least one embodiment of the invention. The system further includes a user interface adapter 19 that connects a keyboard 15, mouse 17, speaker 24, microphone 22, and/or other user interface devices such as a touch screen device (not shown) to the bus 12 to gather user input. Additionally, a communication adapter 20 connects the bus 12 to a data processing network 25, and a display adapter 21 connects the bus 12 to a display device 23 which may be embodied as an output device such as a monitor, printer, or transmitter, for example.

Population Data

In the system and method described above, one component of the risk model is the integration of population data which will be described in further detail below. The present disclosure provides models that leverage such population data to provide a more precise qualitative myopia risk metric.

Fixed Variables

As previously noted, fixed variables are ones that may vary across a population, but are fixed relative to a given subject. One or more models (e.g., formulas) of the present disclosure may be based on fixed variables that may have an effect on the incidence and/or progression of myopia in a subject such as a child under 18 years of age. Such fixed risk factors may comprise age, ethnicity, and the number of myopic parents.

Age

The models of the present disclosure may be based on a baseline estimate of the annual incidence of myopia as a function of age. As an illustrative example, data from Collaborative Longitudinal Evaluation of Ethnicity and Refractive Error (CLEERE) Study which is a US-based study including a good size population and ethnic diversity.

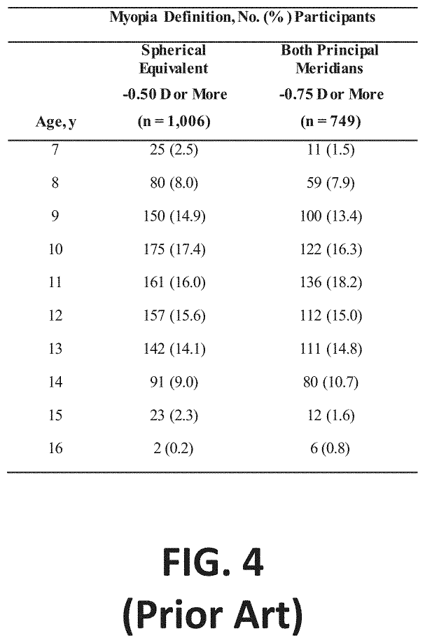

Between 1989 to 2009, a total of 4,927 children 5 to 16 years of age participated in the CLEERE Study. A total of 61.1% of the sample was at least 13 years old at the last study visit. Of the 4,290 of 4,927 children entering the study who were not myopic, 1,006 (22%) became myopic, based on a spherical equivalent of at least -0.50 D. FIG. 4 shows prior art example data on the youngest age at which myopia was observed. Around 15% of cases occur each year between 9 and 13 years. The data were used to calculate the annual incidence of myopia, based on 4,290 subjects. The incidence is around 3.5 to 4% between 9 and 13 years. Data outside this range should be treated with caution as the mean age at study entry was 8.5 years and the mean age at study exit was 12.6 years. Furthermore, 637 children were at least -0.50 D myopic at study entry, suggesting that around 150 children per year might become myopic between the ages of 6 and 8 years.

Table 1 shows incidence rate based on age.

TABLE 1

| [symbol] ge (years) | [symbol] n = 1,006) | ncidence |
|---|---|---|
| | 5 | .58% |
| | 0 | .86% |
| | 50 | .50% |
| 0 | 75 | .08% |
| 1 | 61 | .75% |
| 2 | 57 | .66% |
| 3 | 42 | .31% |
| 4 | 1 | .12% |
| 5 | 3 | .54% |
| 6 | | .05% |

[symbol]

indicates data missing or illegible when filed

The overall impression is that a fairly constant number of children become myopic each year and that this number is close to 4%.

The age dependence of myopia onset can be informed by studies from Asia. In the Singapore Cohort Study of the Risk Factors for Myopia (SCORM), 981 children aged 7 to 9 years were followed up over a 3-year period. Of these, 569 not myopic at baseline had 3-year follow-up data. The 3-year cumulative incidence rate was 47.7% (95% CI: 42.2-53.3) for 7-year-olds, 38.4% (95% CI: 31.4-45.4) for 8-year-olds, and 32.4% (95% CI: 21.8-43.1) for 9-year-olds. The difference was not significant (p=0.057).

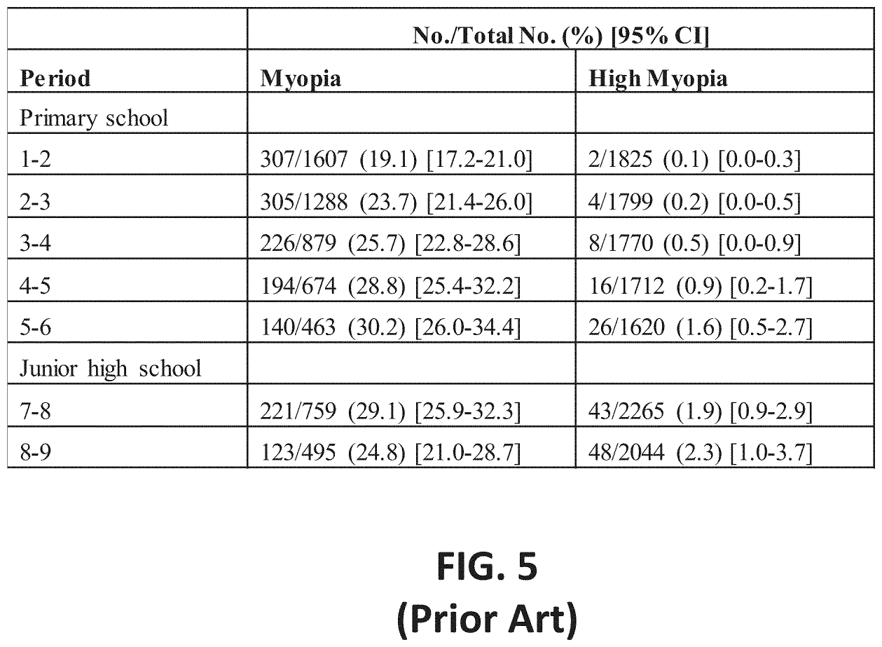

A larger cohort study in Guangzhou, China enrolled 4,741 children in either grade 1 (mean age=7.3 years) or grade 7 (mean age=13.2 years). The younger, primary school cohort were followed for five years and the older, junior high school cohort. FIG. 5 shows prior art example data of annual incidence of myopia and high myopia in primary and junior high school cohorts based on refraction without cycloplegia. At study entry, 1,607 of the younger cohort were nonmyopic. Five years later 1,172 (72.9%) had developed myopia. The incidence of myopia was 20% to 30% each year throughout both cohorts. Note that these incidence estimates are based on the surviving nonmyopes from the previous year, so the incidence decreases with age observe the numerator in the first column.

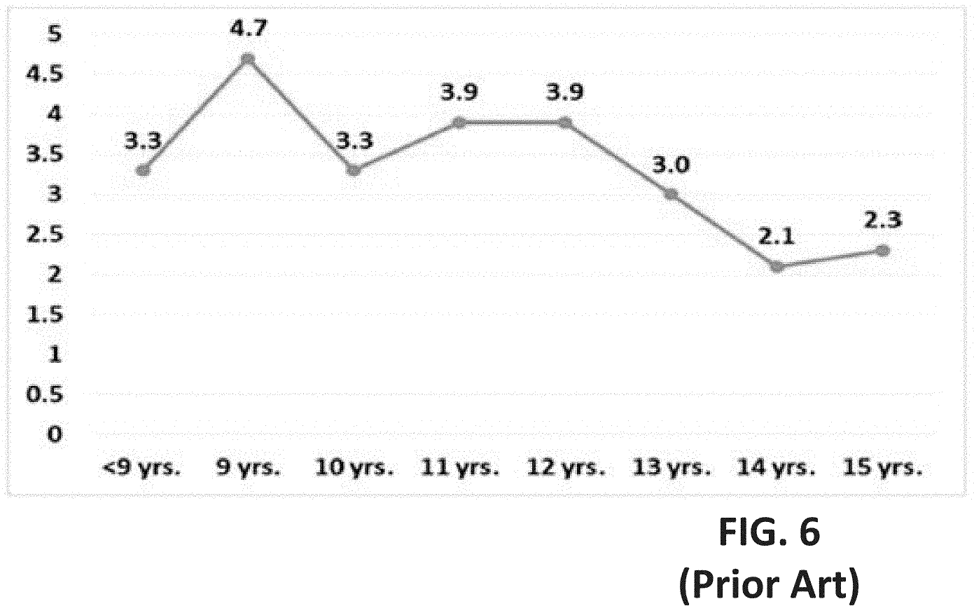

A cohort of 10,000 Indian school children aged 5 to 15 years were recruited and reexamined a year later. Of the 9,616 children completing follow up (97.3%), the incidence of myopia was 3.4%. FIG. 6 shows prior art example plot of age specific incidence of myopia indicating that incidence is relatively constant with age.

The Sydney Adolescent Vascular and Eye Study examined 863 children 6 years after initial examination at a mean age of 6.7 years. A group of 1,196 older children were examined 4.5 years after initial examination at a mean age of 12.7 years. The annual incidence of myopia was 2.2% in the younger cohort and 4.1% in the older children.

Ethnicity

The models of the present disclosure may be based on a baseline estimate of the annual incidence of myopia as a function of ethnicity. As an example, a systematic review identified 143 population-based surveys with estimates of childhood myopia prevalence, representing 42 countries and 374,349 subjects. East Asians showed the highest prevalence, reaching 69% at 15 years of age (86% among Singaporean-Chinese). Blacks in Africa had the lowest prevalence; 5.5% at 15 years. Time trends in myopia prevalence over the last decade were small in whites, increased by 23% in East Asians, with a weaker increase among South Asians.

The above data from different countries show a dramatic variation in annual incidence from 2 to 4% in Australia and India to 10 to 30% in Singapore and China. These may be partly due to lifestyle and educational differences, so it is useful to revisit the aforementioned CLEERE Study.

Of the 4,290 children entering the study who were not myopic, 1,006 (22%) became myopic, based on a spherical equivalent of at least -0.50 D. New cases of myopia occurred in 35% of Asians, 30% of Hispanics, 21% of Native Americans, 22% of African Americans, and 17% of whites. Table 2 shows reconstructed logistic model data with the addition of years of cumulative incidence and annual incidence. The annual incidence is highest in Asian children, but still well below the values in studies conducted in East Asia. The incidence in Hispanics is also higher than in whites. A curious feature of the data in the table is that the annual incidence is higher than that for the paper's other presentation.

Table 2 shows: Incidence of Myopia Each Ethnic/Racial Group

| | | [symbol] ew | [symbol] ge at | [symbol] ge at | [symbol] ears of | | |
|---|---|---|---|---|---|---|---|
| | [symbol] emained | Cases of | Entry | Exit | Follow- | [symbol] umulative | [symbol] nnual |
| [symbol] thnicity | [symbol] Nonmyopic | Myopia | (years) | (years) | up | Incidence | Incidence |
| [symbol] ative | [symbol] 61 | [symbol] 6 | [symbol] .35 | [symbol] 3.39 | [symbol] .04 | [symbol] 1.0% | [symbol] .20% |
| American | | | | | | | |
| [symbol] sian | [symbol] 69 | [symbol] 02 | [symbol] .06 | [symbol] 1.59 | [symbol] .53 | [symbol] 5.4% | [symbol] 0.02% |
| [symbol] frican | [symbol] 48 | [symbol] 55 | [symbol] .17 | [symbol] 3.34 | [symbol] .17 | [symbol] 2.1% | [symbol] .29% |
| American | | | | | | | |
| [symbol] ispanic | [symbol] 55 | [symbol] 76 | [symbol] .76 | [symbol] 2.7 | [symbol] .94 | [symbol] 9.6% | [symbol] .52% |
| [symbol] hite | [symbol] ,321 | [symbol] 67 | [symbol] .05 | [symbol] 2.38 | [symbol] .33 | [symbol] 6.8% | [symbol] .88% |
| [symbol] ther | [symbol] 9 | [symbol] 0 | [symbol] .33 | [symbol] 2.16 | [symbol] .83 | [symbol] 5.6% | [symbol] .69% |
| [symbol] otal | [symbol] ,283 | [symbol] ,006 | [symbol] .62 | [symbol] 2.59 | [symbol] .97 | [symbol] 3.5% | [symbol] .90% |

[symbol]

indicates data missing or illegible when filed

Studies of myopia incidence in multi-ethnic populations are rare, although in the Sydney study, children of East Asian ethnicity had a higher annual incidence of myopia (younger 6.9%, older 7.3%) than those of European descent (younger 1.3%, older 2.9%). Studies of myopia incidence show much higher values as discussed above.

Parental History

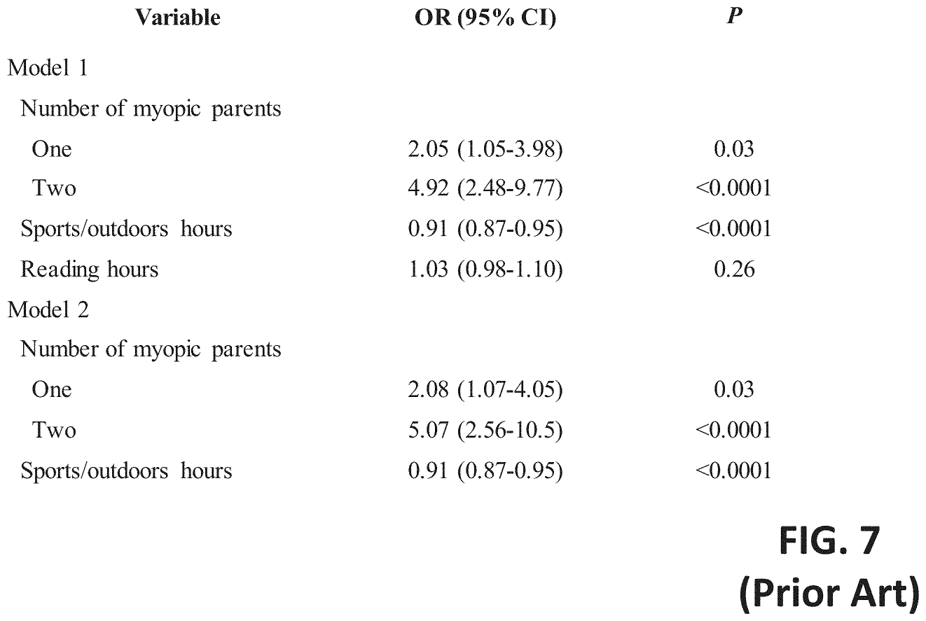

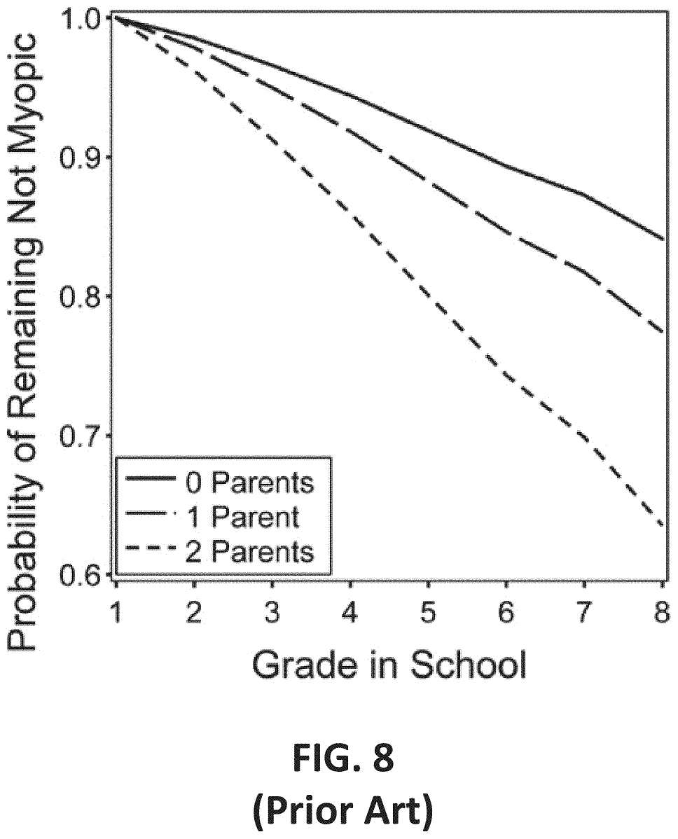

The models of the present disclosure may be based on a baseline estimate of the annual incidence of myopia as a function of genetics such as parental history. It is unequivocal that a parental history of myopia increases the risk of a child becoming myopic. It is unclear whether the mechanism is genetic, due to a shared environment, or a combination. FIGS. 7-9 show prior art data relating to parental history.

The Orinda Longitudinal Study of Myopia analyzed data from 514 non-myopic 3^{rd} grade children (mean age 8.6 years) to predict myopia through 8^{th} grade. Of these 111 (21.6%) became myopic. Parental history of myopia was an important predictor in univariate and multivariate models. In both multivariate models, one myopic parent was associated with a two-fold increase in the odds of developing myopia and two myopic parents a five-fold increase.

A subsequent paper used a larger and more diverse cohort from the CLEERE Study to determine the utility of a child's first grade refractive error and parental history of myopia as predictors of myopia onset between the second and eighth grades. Of the 1,854 nonmyopic first graders, 334 had become myopic by grade 8. The hazard ratio (HR) comparing subjects who had one myopic parent with those with none was 1.48 (95% CI, 1.09-1.99, p=0.01). Children of two myopic parents had an increased hazard ratio of eventual myopia compared with children who had no myopic parents (HR, 2.38; 95% CI, 1.66-3.41; P<0.0001). Note that the ratios in this paper are lower than those in the previous study and largely replicated in a subsequent paper on the same dataset. Overall, age at myopia onset was very similar across the number of myopic parents, although Asian children with no myopic parents develop myopia later than those with at least one myopic parent.

A cross-sectional study of 661 12- to 13-year-old white children in Northern Ireland found a stronger influence of parental myopia. Compared to children with no myopic parents, children with one or two myopic parents were 2.91 times (95% CI, 1.54-5.52) and 7.79 times (95% CI, 2.93-20.67) more likely to have myopia, respectively. The effect of parental myopia remained after adjustment for other factors. The influence of parental myopia was not explored in the authors' subsequent longitudinal study.

Another cross-sectional study analyzed data from 4,677 randomly selected students (mean age 16.9 years; range: 16−18 years) in Beijing. Prevalence of myopia (≤−1.00 D in worse eye) was 80.7%. In multiple logistic regression analysis, a higher prevalence of myopia was associated with one myopic parent (OR=2.28; 95% CI: 1.80-2.87) and two myopic parents (OR=4.02; 95% CI: 2.42-6.66).

Modifiable Risk Factors

One or more models (e.g., formulas) of the present disclosure may be based on modifiable risk factors (e.g., behavioral risk factors) that may have an effect on the incidence and/or progression of myopia in a subject such as a child under 18 years of age. Such fixed risk factors may include the time per day of outdoor activity and the time per day of nearwork, for example.

Outdoor Activity

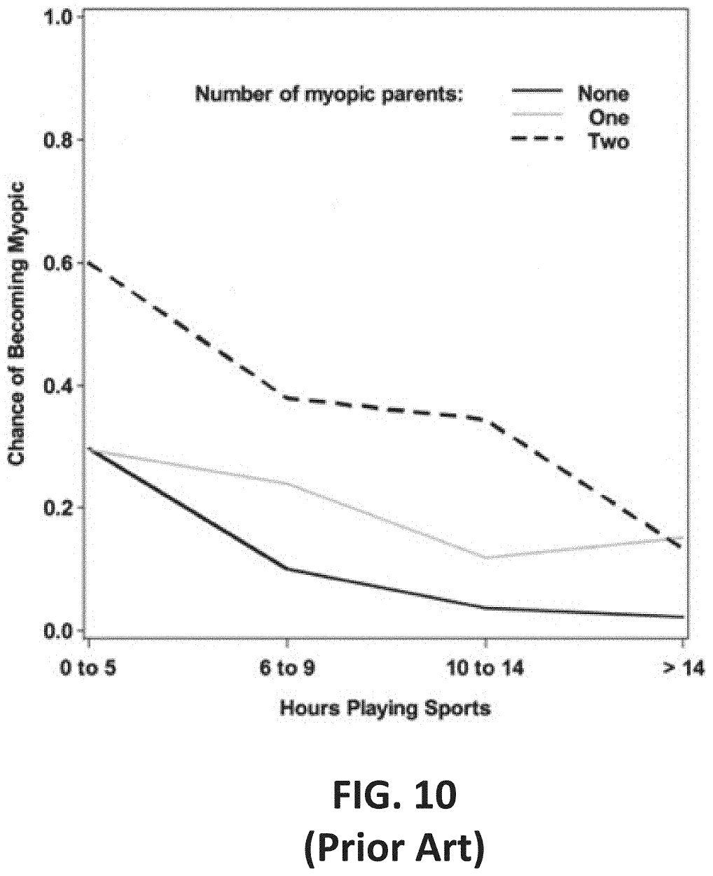

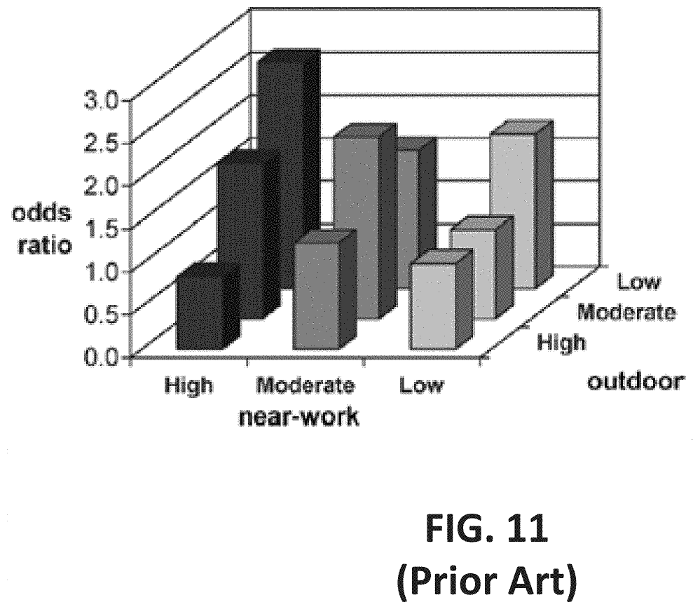

The models of the present disclosure may be based on a baseline estimate of the annual incidence of myopia as a function of time a subject spends on outdoor activity. A number of studies in the US, Australia, Singapore, UK and Taiwan have reported a robust relation between outdoor activity and myopia. FIGS. 10-11 show prior art data relating to modifiable risk factors.

The Orinda Longitudinal Study of Myopia analyzed data from 514 nonmyopic 3^{rd} grade children (mean age 8.6 years) to predict myopia through 8^{th} grade. Of these 111 (21.6%) became myopic. In both multivariate models, higher amounts of outdoor activity (hours per week) was associated decrease in the odds of developing myopia. The effect is present regardless of the number of myopic parents. The odds ratio of 0.91 per hour each week corresponds to around 0.5 per hour each week. In other words, each additional daily hour of outdoor activity halves the risk of myopia onset. A significant interaction in the logistic model showed a differential effect of sport and outdoor activity hours per week based on a child's number of myopic parents.

A similar finding was reported from a cross-sectional study of randomly sampled Sydney schoolchildren: 1,965 6-year-olds and 2,367 12-year-olds. Higher levels of outdoor activity were associated with more hyperopic refractions and lower myopia prevalence in the 12-year-old students. The study shows the influence of outdoor activity on the presence of myopia, although the influence of nearwork is less obvious.

The follow up Sydney Adolescent Vascular and Eye Study examined 863 young children (mean age 6.7 years)6 years after initial examination. A group of 1,196 older children (mean age 12.7 years) were examined 4.5 years after initial examination. Children who became myopic spent less time outdoors compared with children who remained nonmyopic (younger cohort, 16.3 vs. 21.0 hours, respectively, P<0.0001; older cohort, 17.2 vs. 19.6 hours, respectively, P 0.0.001). In the younger cohort, children with <16 hours per week of outdoor activity were more likely to develop myopia than those who spent >23 hours per week outdoors (odds ratio 2.84; 95% CI 1.56-5.17). Likewise, in the older cohort, children with <13.5 hours per week of outdoor activity were more likely to develop myopia than those who spent >22.5 hours per week outdoors (odds ratio 2.35; 95% CI 1.30-4.27).

The relationship between outdoor activities and myopia was confirmed in a cross-sectional study of 1,249 teenage Singaporean children. The total outdoor activity in hours per day was significantly associated with myopia (odds ratio 0.90, 95% CI 0.84 to 0.96), after adjusting for age, gender, ethnicity, school type, books read per week, height, parental myopia, parental education and intelligence quotient. Total sports was also significantly negatively associated with myopia (p=0.008) but not indoor sports (p=0.16).

The Avon Longitudinal Study of Parents and Children (ALSPAC) assessed participants at ages 7, 10, 11, 12, and 15 years (N=4,837 to 7,747). Physical activity at age 11 years was measured objectively using an accelerometer, worn for 1 week. Time spent outdoors was assessed via a parental questionnaire administered when children were aged 8−9 years asking “On a (weekend day)/(school week day), how much time on average does your child spend each day out of doors in (summer)/(winter). “The authors selected the variable one that displayed the strongest association with outcomes of interest: time spent outdoors on a weekend day in summer, where a ‘high’ amount was 3 or more hours per day and as low was less than 3. Both time spent outdoors and physical activity were associated with incident myopia, with time outdoors having the larger effect. For nonmyopic children at age 11, the hazard ratio for incident myopia was 0.66 (95% CI 0.47-0.93) for a high versus low amount of time spent outdoors.

Finally, the Myopia Investigation Study in Taipei was a citywide, population-based cohort study that enrolled 11,590 grade 2 schoolchildren (−8 years). Among baseline nonmyopic participants, 6,794 were examined during the first-year follow-up, of whom 1,856 (25.2%) had developed myopia were identified. Protective factors included suburban area of residence (HR: 0.91; 95% CI: 0.83-1.00) and spending at least 30 minutes outdoors after school every weekday was protective against myopia (HR: 0.90; 95% CI: 0.82-0.99).

In the past decade, there have been a number of randomized clinical trials to evaluate the effect of additional outdoor activity on the incidence of myopia. The first such trial randomized 7 to 11 year old Taiwanese children to an intervention (n=333) where they were encouraged to go outside for outdoor activities during recess or a control (n=238) where there was no such programs during recess. After 1 year, the incidence of myopia was significantly lower in the intervention group than in the control group (8.4% vs. 17.6%; P<0.001). The total daily recess time was 80 minutes (10, 20, and 10 minutes in both the morning and afternoon), and the total weekly recess time was approximately 6.7 hours, an average of around 1 hour per day.

The same group reported a second clinical trial where the intervention group was encouraged to go outdoors for up to 11 hours weekly. A total of 693 students in 16 schools completed the full 1-year program-267 in the intervention group and 426 in the control group. Among the 620 nonmyopic children at baseline, the incidence of myopia in the intervention group was less than that in the control group (14.47% vs. 17.40%; odds ratio, 0.65; 95% CI, 0.42-1.01). The smaller effect may be explained by a number of factors. After the initial randomization, half of the 16 randomly selected schools withdrew from the program. Also, Taiwan had introduced a national myopia prevention program in schools that aims for two hours per day outdoors, more than the intervention. It also has a program that aims for 150 minutes of exercise at school per week.

A similar randomized clinical trial of grade 1 children (mean age 6.6 years) was conducted in Guangzhou, China. For six intervention schools (n=952), an additional 40 minutes of outdoor activities was added to each school day, and parents were encouraged to engage their children in outdoor activities after school hours. Children in the six control schools (n=951) continued their usual pattern of activity. The 3-year incidence of myopia was 30.4% in the intervention group (259 of 853 nonmyopic participants) and 39.5% (287 of 726 participants) in the control group (difference=−9.1%; 95% CI, −14.1% to −4.1%).

Measurable Risk Factors

One or more models (e.g., formulas) of the present disclosure may be based on measurable risk factors that may have an effect on the incidence and/or progression of myopia in a subject such as a child under 18 years of age. Such measurable risk factors may include refractive error or axial length of an eye of a subject, for example.

Refractive Error

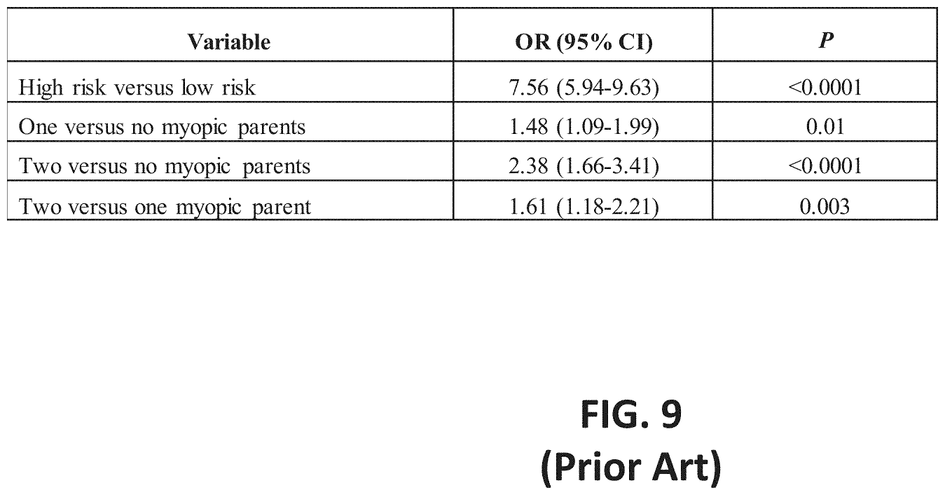

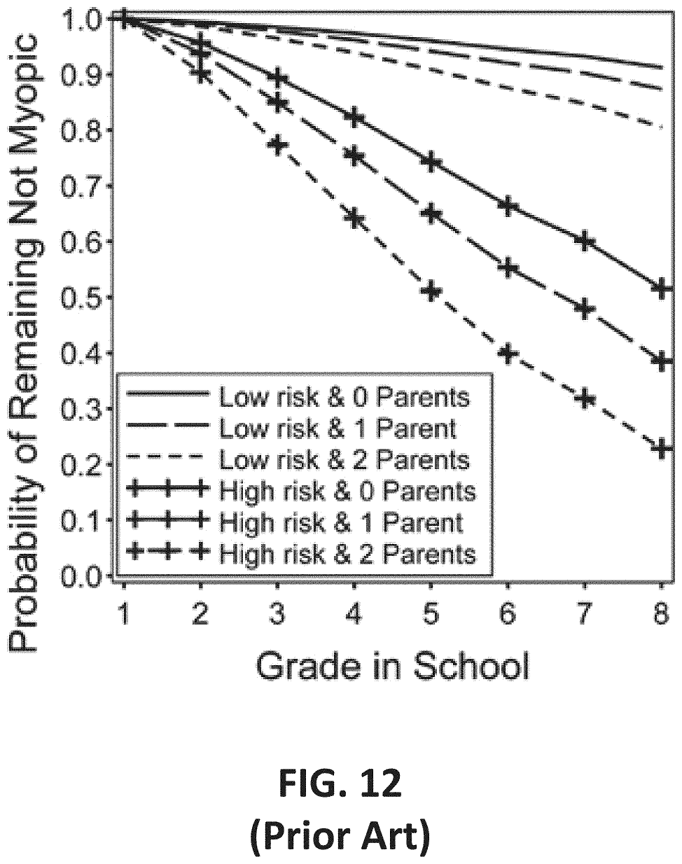

The CLEERE Study determined the utility of a child's first grade refractive error and parental history of myopia as predictors of myopia onset between the second and eighth grades. Based on previous work, children were classified into high- and low-risk myopia groups. High risk of myopia among nonmyopic children was defined as +0.75 D or less in the more hyperopic meridian in the first grade. Of the 1,854 nonmyopic first graders, 334 had become myopic by grade 8. Overall, 21.3% of the first graders fell into the high-risk group. FIG. 12 shows survival probability curves for no, one, and two myopic parents as a function of risk group. FIG. 9 shows hazard ratio (HR) for myopia incidence. The hazard ratio for the incidence of myopia given the high-risk category was 7.56 (95% CI, 5.94-9.63). Note that this is substantially higher than the risk associated with having two myopic parents. The model estimates for Asians and whites were similar to those for the group in its entirety.

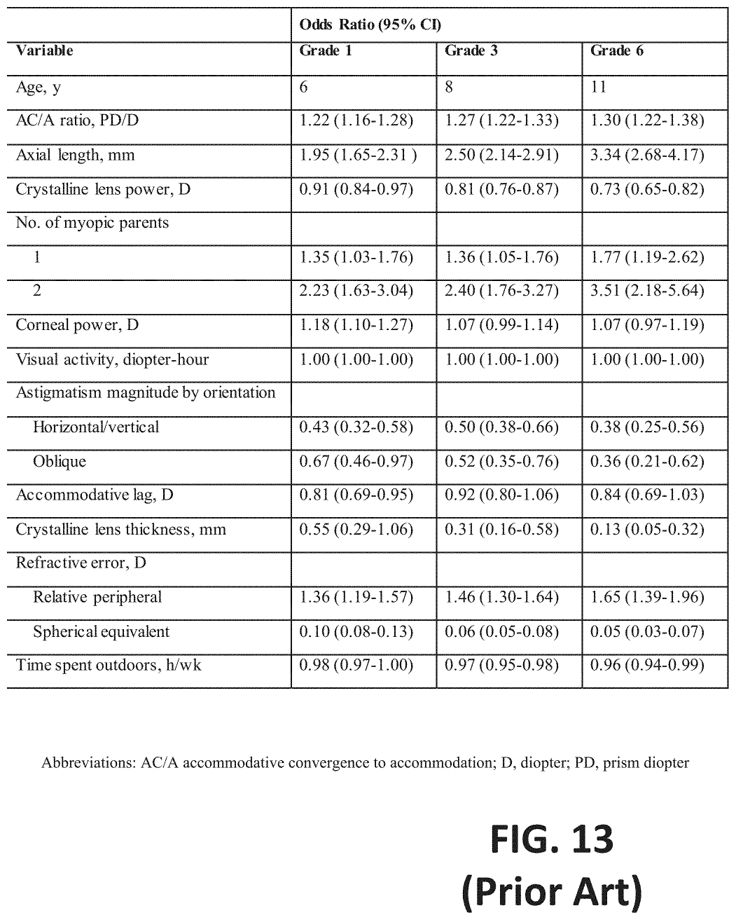

A subsequent paper analyzed a larger sample of 4,512 ethnically diverse, nonmyopic children from grades 1 through 8 (baseline, 6 through 11 years). FIG. 13 shows univariate analysis of risk factors for developing myopia. Multivariant analysis was also conducted. A total of 414 children became myopic from grades 2 through 8 (ages 7 through 13 years). Of the 13 factors evaluated, 10 were associated with the risk for myopia onset (P<0.05) and 8 retained their association in multivariate models: spherical equivalent refractive error at baseline, parental myopia, axial length, corneal power, crystalline lens power, ratio of accommodative convergence to accommodation (AC/A ratio), horizontal/vertical astigmatism magnitude, and visual activity. A less hyperopic/more myopic baseline refractive error was consistently associated with risk of myopia onset in multivariate models (odds ratios from 0.02 to 0.13, P<0.001), while nearwork, time outdoors, and having myopic parents were not. The authors conclude that future myopia can be predicted in a nonmyopic child using a simple, single measure of refractive error. Future trials for prevention of myopia should target the child with low hyperopia as the child at risk.

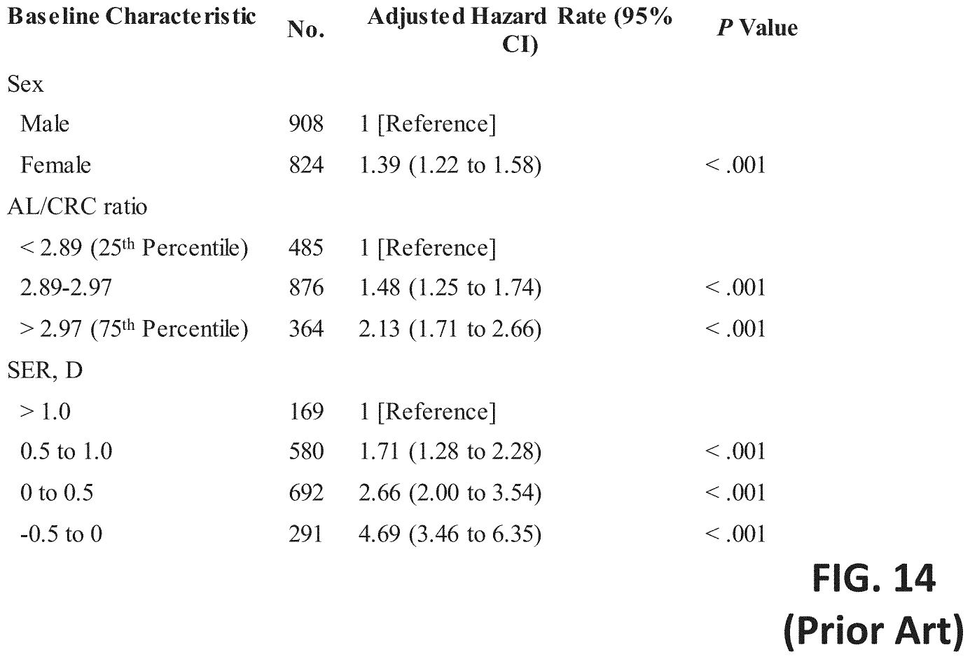

Studies in China support the importance of a hyperopic buffer. A cohort study in Guangzhou, China recruited 1,975 students in grade 1 (mean age 7.2 years) and 2,670 students in grade 7 (mean age 13.2 years). The younger cohort was followed for five years and the older cohort two years. Baseline prevalence of myopia was 12.0% in grade 1 students (n=237 of 1969) and 67.4% in grade 7 students (n=1795 of 2663). The annual incidence of myopia was 20% to 30% in both cohorts. An extract of the data presented in the study is shown in FIG. 14, demonstrating the protective effect of increasing levels of hyperopia at baseline.

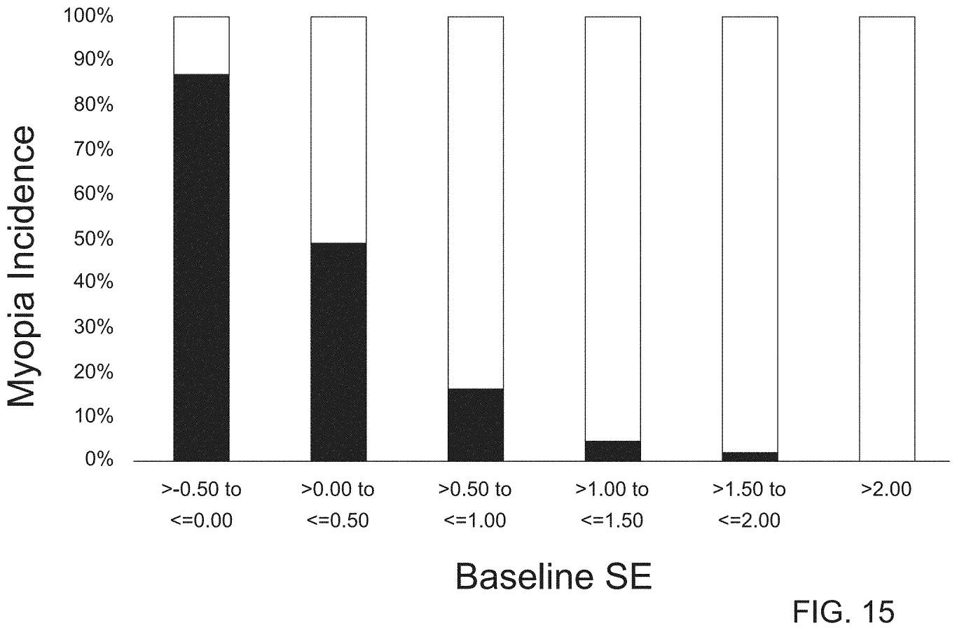

A similar cohort study in Shanghai recruited 1,856 students in grades 1 through 3 (mean ages 7.1, 8.1, and 9.2 years of whom 1,567 were nonmyopic at baseline and 1,385 were reexamined two years later). Only parental myopia, but not nearwork time, nearwork, outdoor activity time or attending tutoring classes, was associated with myopia incidence. The relation between baseline refractive error and the incidence of myopia is shown in FIG. 15. The best predictor of 2-year incidence of myopia was spherical equivalent of +0.50 D or less with a sensitivity of 85% and a specificity of 71%. If specificity was set at >80%, spherical equivalent of +0.37 D or less was the best predictor with sensitivity of 75%.

Risk of High Myopia

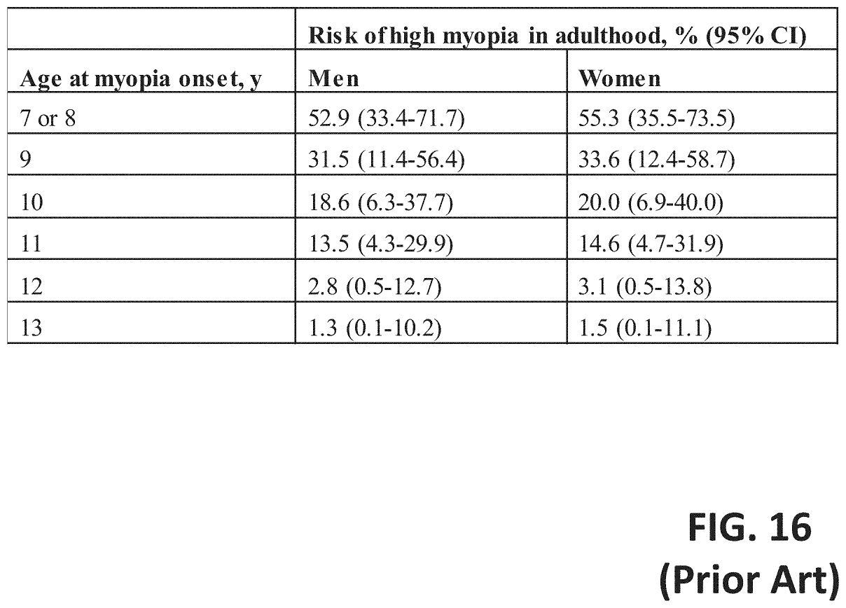

One or more models (e.g., formulas) may be used to provide a myopia risk factor. As an example, the probability of high myopia (at least -5 D) may be predicted for onset at each age (e.g., prior to 18 years of age). The models of the present disclosure may be based on risk factors such as age of onset and ethnicity. While myopia onset younger than 8 years is less common, the risk of the child progressing to high myopia is greater. A study of 443 Chinese children who developed myopia found that for myopia onset at 7 or 8 years, 54% developed high myopia by adulthood. In contrast, only 19% of those with onset at 10 years of age developed high myopia. FIG. 16 shows risk of high myopia data. An earlier study of Singaporean children which only studied subjects through 11 years, but 87% of those who developed high myopia had an age of onset at 7 years or younger.

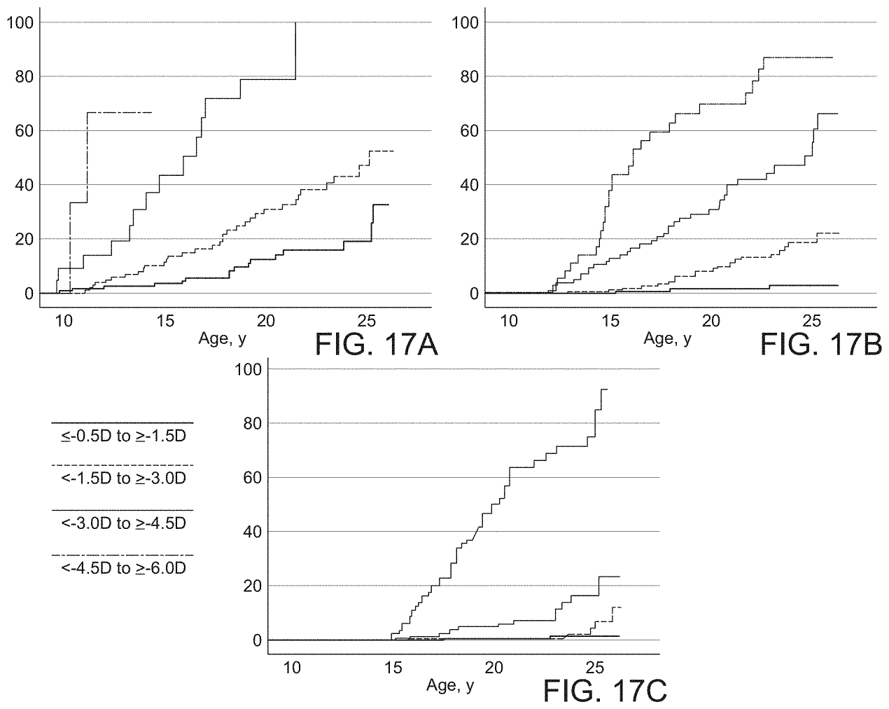

The Drentse Refractive Error and Myopia (DREAM) Study reported progression data on 2,555 myopes using retrospective data from a branch of opticians in the Netherlands. Subjects with prescriptions at an interval of at least 1 year were included in the analysis. Those with first prescription before the age of 10 years showed the fastest progression with a median spherical equivalent of -4.48 D (IQR: -5.37 to -3.42 D). FIGS. 17A-17C illustrates the risk of developing high myopia as a function of age. All children who were at least -3 D at 10 years were highly myopic (at least -6 D) as adults. Children between -1.50 and −3.00 D at 10 years had a 46.0% risk of high myopia, and children between −0.50 and −1.50 D had a 32.6% risk. These values are for 25 years of age. The corresponding percentages for high myopia at 18 years are 71.7%, 21.4% and 5.5%.

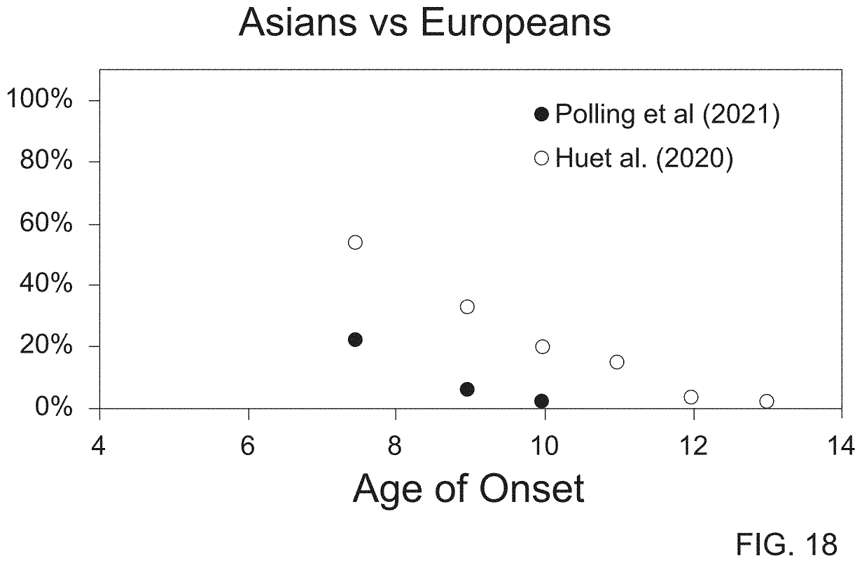

FIG. 18 shows a comparison of the risk of high myopia as a function of age of onset in Asian and European myopes. The risk of high myopia is very different due to the higher annual progression in Asian eyes. The data for European myopes appear shifted about 2.5 years to the left.

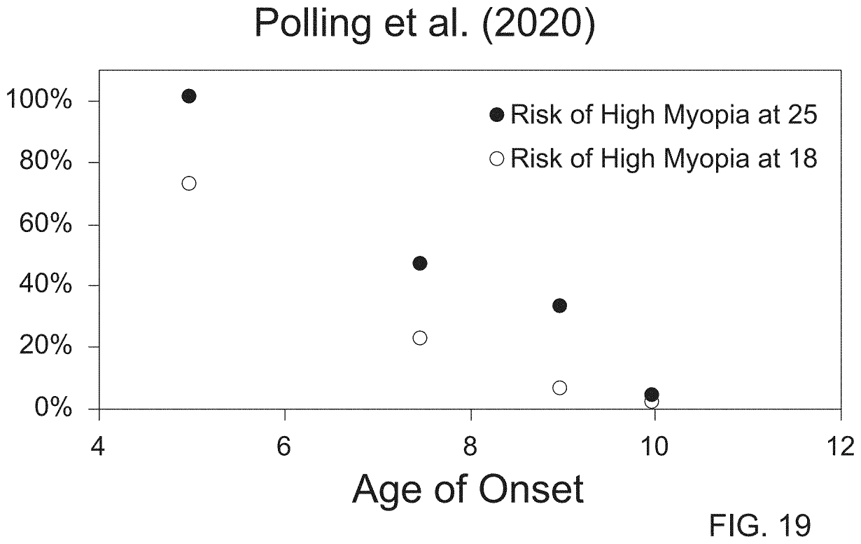

FIG. 19 shows a comparison of the risk of high myopia at 25 years and 18 years as a function of age of onset in European myopes. For myopia onset prior to the age of 10 years, the risk of myopia increases by around 30% between the ages of 18 and 25 years.

An example model for a myopia risk calculator is described herein. As an example, an annual incidence may be calculated, which is considered constant from the child's current age+1 through 18 years. For example:

Annual incidence=Baseline Incidence ×(1+Gender×0.15)×Ethnicity× 1.6^{#myopic parents-1}

Where:

Baseline Incidence=0.04 or 4%

Gender=1 for female, 0 for male

Ethnicity=2.5 for Asian, 2 for Hispanic, 1 for others

Note: calculate for years beyond age

If data on nearwork hours and outdoor activities available:

Annual incidence=Baseline Incidence ×(1+Gender×0.15)×Ethnicity× 1.6#^{myopic parents-1}×0.5^{Outdoors-1}×1.1 ^{Nearwork-1}

Where:

Outdoors=Number of hours per day spent outdoors (=1 if unknown)

Nearwork=Number of hours per day spent on nearwork (=1 if unknown)

A cumulative incidence may be calculated, which is considered constant from the child's current age through 18 years.

Cumulative incidence may be equal to: Previous year cumulative incidence+Annual Incidence ×(1 - Previous year cumulative incidence)

Probability of high myopia (at least −6 D) may be predicted for onset at each age:

If Ethnicity=Asian

Probability of high myopia=Annual Incidence ×10^{2.1-0.293×Age}×(0.9+0.1 ×#myopic parents^{1.5})×(0.98+0.02 ×Gender)

If Ethnicity # Asian

Probability of high myopia=Annual Incidence ×10^{137-0.293Age}×(0.9+0.1 ×#myopic parents”)×(0.98+0.02 ×Gender)

For both Probability of high myopia cannot exceed Annual Incidence:

If Probability of high myopia >Annual Incidence, then Probability of high myopia >Annual Incidence

A cumulative probability of high myopia (at least −5 D) may be determined. As an example, Total Probability of high myopia=Sum of above probabilities at each age

If >0.99, then=0. 99

A cumulative probability of myopia at least −5 D may be determined.

Total Probability of at least −5 D=Total Probability of high myopia x 1.4

If >0.99, then=0. 99.

What is claimed is:

1. A computer-implemented method comprising:
receiving, via an interface, demographic information indicative of an age of a subject, a gender of the subject, an ethnicity of the subject, and a number of myopic parents of the subject;
receiving, via the interface, behavioral information indicative of a time that the subject spends outside each day and a time that the subject spends on nearwork each day;
determining an incidence factor for the subject by weighting, according to a predetermined incidence formula, the demographic information and the behavioral information,
wherein the predetermined incidence formula and weighting is derived from incidence data associated with a population;
determining a progression factor for the subject by weighting, according to a predetermined progression formula, the demographic information and the behavioral information, wherein the predetermined progression formula and weighting is derived from progression data associated with a population, and wherein the predetermined progression formula is a function of the incidence factor;
predicting and calculating, by a processor and based on the incidence factor and the progression factor, a myopia risk metric indicative of risk of the subject exhibiting myopia; and
causing output of the myopia risk metric comprising a quantitative numerical component.

2. The method of claim 1, wherein the incidence factor is based at least on the following formulaic relationship of:
incidence factor=BI ×G×α×E×β^{MP}
where BI is a baseline incidence factor, G is a gender weighting factor, E is an ethnicity weighting factor, MP is the number of myopic parents of the subject, and a and β are evidence-based weighting factors.

3. The method of claim 1, wherein the incidence factor is based at least on the following
formula:
incidence factor=BI ×(1+G×0.15)×E×1.6^{MP-1},
where BI is a baseline incidence factor, G is a gender weighting factor, E is an ethnicity
weighting factor, and MP is the number of myopic parents of the subject.

4. The method of claim 3, wherein BI is 0.04.

5. The method of claim 3, wherein G is 1 for female and 0 for male.

6. The method of claim 3, wherein E is 2.5 for Asian, 2 for Hispanic, 1 for others.

7. The method of claim 1, wherein the incidence factor is based at least on the following formula:
incidence factor=BI ×(1+G×0.15)×E×1.6^{MP-1}×0.5^{OT-1}×1.1^{NT-1}
where BI is a baseline incidence factor, G is a gender weighting factor, E is an ethnicity weighting factor, MP is the number of myopic parents of the subject, OT is the time (hours) the subject spends outside each day, and NT is the time (hours) the subject spends on nearwork each day.

8. The method of claim 7, wherein BI is 0.04.

9. The method of claim 7, wherein G is 1 for female and 0 for male.

10. The method of claim 7, wherein E is 2.5 for Asian, 2 for Hispanic, 1 for others.

11. The method of claim 1, wherein the progression factor indicates a probability of the subject exhibiting high myopia, wherein high myopia is one of at least −4D or at least −6D.

12. The method of claim 1, wherein the progression factor for a subject having ethnicity of Asian is based on at least the following formula:
progression factor=incidence factor x 10^{2.1-0.293×A} ×(0.9+0.1 ×MP^{1.5})×(0.98+0.02 ×G),
where A is age in years of the subject, G is a gender weighting factor, and MP is the number of myopic parents of the subject.

13. The method of claim 12, wherein G is 1 for female and 0 for male.

14. The method of claim 1, wherein the progression factor for a subject having ethnicity of non-Asian is based on at least the following formula:
progression factor=incidence factor ×10^{1.37-0.293×A} ×(0.9+0.1 ×MP^{1.5})×(0.98+0.02 ×G),
where A is age in years of the subject, G is a gender weighting factor, and MP is the number of myopic parents of the subject.

15. The method of claim 14, wherein G is 1 for female and 0 for male.

16. The method of claim 1, further comprising receiving diagnostic information indicative of one or more of a refractive error associated with the subject or axial length of an eye of the subject indicating that the subject is non-myopic, wherein one or more of the incidence factor or the progression factor is determined based on at least the diagnostic information.

17. The method of claim 1, wherein the risk metric comprises a severity metric.

18. The method of claim 17, wherein the severity metric comprises a projected level of myopia.

19. The method of claim 1, wherein the quantitative numerical component is a percentage.

20. A device configured to implement the method of claim 1.

21. A system configured to implement the method of claim 1.

22. A computer-implemented method comprising:
receiving, via an interface, demographic information of a subject receiving, via the interface, behavioral information of the subject;
determining, based on one or more of the demographic and behavioral information or the second information, an incidence factor for the subject by weighting, according to a predetermined incidence formula, the demographic information and the behavioral information,
wherein the predetermined incidence formula and weighting is derived from incidence data associated with a population;
determining, based on one or more of the demographic information or the behavioral information, a progression factor for the subject by weighting, according to a predetermined progression formula, the demographic information and the behavioral information, wherein the predetermined progression formula and weighting is derived from progression data associated with a population, and wherein the predetermined progression formula is a function of the incidence factor;
predicting and calculating, by a processor and based on the incidence factor and the progression factor, one or more of a myopia risk metric indicative of risk of the subject exhibiting myopia or a severity metric indicative of a level of myopia; and
causing output of the one or more of the myopia risk metric or the severity metric,
wherein each of the myopia risk metric and the severity metric comprise a quantitative numerical component.

23. The method of claim 22, wherein the first information comprises an age of the subject.

24. The method of claim 22, wherein the first information comprises a gender of the subject.

25. The method of claim 22, wherein the first information comprises an ethnicity of the subject.

26. The method of claim 22, wherein the first information comprises a number of myopic parents of the subject.

27. The method of claim 22, wherein the second information comprises a time that the subject spends outside each day.

28. The method of claim 22, wherein the second information comprises a time that the subject spends on nearwork each day.

29. The method of claim 22, further comprising receiving, via the interface, measurable diagnostic information indicative of one or more of a refractive error associated with the subject or axial length of an eye of the subject, wherein the predicting and calculating a myopia risk metric indicative of risk of the subject exhibiting myopia is based at least on the third information.

30. The method of claim 22, wherein the incidence factor is based at least on the following formulaic relationship of:
incidence factor=BI ×G×α×E×β^{MP},
where BI is a baseline incidence factor, G is a gender weighting factor, E is an ethnicity weighting factor, MP is the number of myopic parents of the subject, and a and ii are evidence-based weighting factors.

31. The method of claim 22, wherein the incidence factor is based at least on the following
formula:
incidence factor=BI ×(1+G×0.15)×E×1.6^{MP-1},
where BI is a baseline incidence factor, G is a gender weighting factor, E is an ethnicity weighting factor, and MP is the number of myopic parents of the subject.

32. The method of claim 31, wherein BI is 0.04.

33. The method of claim 31, wherein G is 1 for female and 0 for male.

34. The method of claim 31, wherein E is 2.5 for Asian, 2 for Hispanic, 1 for others.

35. The method of claim 22, wherein the incidence factor is based at least on the following
formula:
incidence factor=BI ×(1+G×0.15)×E×1.6^{MP-1} ×0.5^{OT-1} ×1.1^{NT-1}
where BI is a baseline incidence factor, G is a gender weighting factor, E is an ethnicity
weighting factor, MP is the number of myopic parents of the subject, OT is the time (hours) the subject spends outside each day, and NT is the time (hours) the subject spends on nearwork each day.

36. The method of claim 35, wherein BI is 0.04.

37. The method of claim 35, wherein G is 1 for female and 0 for male.

38. The method of claim 35, wherein E is 2.5 for Asian, 2 for Hispanic, 1 for others.

39. The method of claim 22, wherein the progression factor indicates a probability of the subject exhibiting high myopia (at least −6D) through 18 years of age of the subject.

40. The method of claim 22, wherein the progression factor for a subject having ethnicity of Asian is based on at least the following formula:
Progression factor=incidence factor ×10^{2.1-0.293×A}×(0.9+0.1 ×MP^{1.5})×(0.98+0.02 ×G),
where A is age in years of the subject, G is a gender weighting factor, and MP is the number of myopic parents of the subject.

41. The method of claim 40, wherein G is 1 for female and 0 for male.

42. The method of claim 22, wherein the progression factor for a subject having ethnicity of non-Asian is based on at least the following formula:
Progression factor=incidence factor ×10^{1.37-0.293×A}×(0.9+0.1 ×MP^{1.5})×(0.98+0.02 ×G),
where A is age in years of the subject, G is a gender weighting factor, and MP is the number of myopic parents of the subject.

43. The method of claim 42, wherein G is 1 for female and 0 for male.

44. The method of claim 22, wherein the myopia severity metric comprises a projected level of myopia.

45. The method of claim 22, wherein the risk metric comprises a severity metric.

46. The method of claim 45, wherein the myopia severity metric comprises a projected level of myopia.

47. The method of claim 22, wherein the quantitative numerical component is a percentage.

48. A device configured to implement the method of claim 22.

49. A system configured to implement the method of claim 22.
